(19) United States
(12) Patent Application Publication
(10) Pub. No.: US 2010/0099135 A1
(43) Pub. Date: Apr. 22, 2010

(54) METHODS AND ASSAYS FOR ASSESSING THE QUALITY OF EMBRYOS IN ASSISTED REPRODUCTION TECHNOLOGY PROTOCOLS

(76) Inventors: Mandy Katz-Jaffe, Denver, CO (US); David K. Gardner, McKinnon (AU)

Correspondence Address: FOLEY & LARDNER LLP, 150 EAST GILMAN STREET, P.O. BOX 1497, MADISON, WI, 53701-1497, US

(21) Appl. No.: 12/603,153

(22) Filed: Oct. 21, 2009

Related U.S. Application Data
(60) Provisional application No. 61/107,401, filed on Oct. 22, 2008.

(51) Int. Cl. C12Q 1/02

(52) U.S. Cl. 435/29

(57) ABSTRACT

The present invention provides methods and assays for assessing the presence, viability and/or developmental stage of an embryo. The present invention also provides method and assays for assessing the quality of an Assisted Reproduction Technology protocol.

### CROSS-REFERENCE TO RELATED APPLICATIONS

This applications claims priority to U.S. Provisional Application No. 61/107,401, filed Oct. 22, 2008, the entire contents of which are hereby incorporated by reference in its entirety.

### TECHNICAL FIELD

The present technology relates to methods for characterizing the secretome of developing embryos and for identifying biomarkers and biomarker patterns indicative of the viability and/or development stage of developing embryos. This invention further relates to methods for improving artificial reproduction protocols based on protein profiles and biomarkers characteristic of natural reproductive processes.

### BACKGROUND

In vitro fertilization (IVF) has become a proven treatment of human infertility with the birth of two million babies. However, worldwide success rates remain low even after 25 years of ongoing advancements in the field. There is a significant loss of human IVF embryos during both the pre and post-implantation stages. Due to this loss most IVF clinics will transfer between 2-4 embryos resulting in the recognized epidemic of high multiple pregnancy rates in human IVF. A deeper understanding of the regulation, formation and development of human gametes and embryos is essential to achieving routine single embryo transfer, higher live birth rates and decreasing multiple pregnancies.

The need to distinguish between viable and non-viable preimplantation embryos is a pre-requisite for moving to single embryo transfers and reducing multiple gestations. Genetic studies on the mammalian preimplantation embryo are providing a wealth of information regarding gene expression and development. However, analyzing genome sequences alone is not likely to lead to new therapies or diagnostic assays. Rather, an understanding of protein production for cellular function and metabolism may be a better process to facilitate the discovery of new diagnostic assays.

The ability to monitor the production of key developmental proteins in a non-invasive fashion should provide a better predictor of embryonic viability than the current use of morphology alone. Ideally, a sensitive non-invasive biochemical assay will directly measure the presence or absence of a protein correlated to embryo viability. From a clinical perspective, this identification of the single most viable embryo among the cohort of embryos available for transfer could result in higher clinical pregnancy rates and a reduction in the incidence of multiple gestations. The desire to achieve routine single embryo transfers, eliminate the danger of multiple pregnancies and increase the live birth rate makes the true assessment of embryo viability an essential tool in human IVF.

### SUMMARY

A first aspect of the present invention provides methods for obtaining protein profiles representative of the secretome of a developing embryo. These protein profiles are obtained from a mass spectrometric analysis of spent media that have come into contact with a developing embryo. Biomarkers that correlate to embryo viability and/or developmental stage may be identified from the protein profiles. These biomarkers may be used in non-invasive clinical assays to assess the viability and or developmental stage of an embryo. These assays may be used to identify the highest quality pre-implantation embryos in an assisted reproduction technology (ART) procedure. This, in turn, allows for the implantation of only the highest quality embryo or embryos, resulting in fewer multiple pregnancies, without sacrificing the success rate of the ART procedure.

As used herein, the term “ART” is used broadly in include all technologies (not just in vitro fertilization (IVF)) that can be utilized in the field of reproduction to assist couples in overcoming infertility. By way of non-limiting example, ART includes all fertility treatments in which both eggs and sperm are handled. ART includes, but is not limited to, procedures that involve surgically removing eggs from a woman's ovaries, combining them with sperm in the laboratory, and returning them to the woman's body or donating them to another woman.

In general, the methods of this first aspect of the invention are conducted according to the following procedure. An analyte sample composed of a spent medium from an ART protocol (e.g., spent media from a pre-implantation IVF procedure) and containing proteins secreted by the cells of an embryo is collected. The analyte sample is then subjected to a mass spectrometric analysis to obtain a protein profile for the sample. The sample protein profile may be compared to a control protein profile obtained for a reference sample to identify, and desirably eliminate, those peaks that do not represent proteins secreted from embryonic cells. The protein profiles may be obtained for embryos in various stages of development for both viable embryos and degenerating embryos. The reference or control sample may be a sample of embryos known to have a good developmental potential or known to result in successful IVF pregnancies. Alternatively, the reference or control sample may be a sample of embryos known to have a poor developmental potential or known not to result in successful IVF pregnancies.

Once the protein profiles have been obtained, biomarkers indicative of embryo viability or developmental stage may be identified by comparing the protein profiles for the different analyte samples to determine whether differences in the patterns of protein expression exist between the samples, thereby identifying biomarkers or biomarker panels. These differences may include differences in the proteins that are present in the sample, or quantitative differences in the relative amounts of the various proteins being expressed. Typically, individual protein profiles are compared, however, it is also possible to compare averaged protein profiles, where an average protein profile for a given sample may be prepared by averaging multiple individual protein profiles taken for that sample. As used herein, the phrase “comparing the protein profiles,” (or similar phrases) includes comparisons between both individual and averaged protein profiles.

In some embodiments, biomarkers indicative of embryo viability are identified by analyzing and comparing one or more samples that contain proteins secreted from the embryonic cells of a viable embryo with one or more samples that contain protein secreted from the embryonic cells of a degenerating embryo. In other embodiments, biomarkers indicative of embryo development are identified by analyzing and comparing different samples that contain proteins secreted from the embryonic cells of embryos in different stages of development. The biomarkers may be stored in a database and used in assays for the assessment of embryos as candidate for use in assisted reproductive technologies, such as IVF.

A second aspect of the present invention provides methods for obtaining protein profiles representative of the proteins present in a natural developing embryo. These protein profiles are obtained from a mass spectrometric analysis of embryos or embryonic tissues from an in vivo developing embryo that is produced via natural reproduction. Biomarkers representative of a naturally produced embryo (i.e., as opposed to an embryo produced via ART) may be identified from the protein profiles. These biomarkers may be used in research and development to assess the quality of, and to improve, ART protocols. In these assays a protein profile for an in vitro embryo developed in an ART procedure is obtained and biomarkers characteristic of an in vivo embryo are identified from the profile. If one or more biomarkers are lacking, one or more parameters for the ART protocol are varied to provide an in vitro embryo having a protein profile that more closely mimics the protein profile of an in vivo embryo. This process may be repeated, changing various aspects of the protocol in a step-wise fashion, until the differences between the protein profile for the in vitro embryo and the protein profile for the in vivo embryo are minimized or eliminated, thereby optimizing the protocol. These methods may be used to assess and improve various aspects of ART protocols including, but not limited to, stimulation protocols (e.g., oocyte stimulation protocols), embryo cryopreservation, embryo transfer and embryo culturing protocols.

### BRIEF DESCRIPTION OF THE FIGURES

FIGS. 1A-1D show mass spectra representative of the protein profiles for human embryos, obtained in accordance with Example 1, below.

FIGS. 2A-2D show mass spectra representative of the protein profiles for mouse embryos, obtained in accordance with Example 2, below.

FIG. 3 is a heat map for the proteins present in an in vivo embryo.

### DETAILED DESCRIPTION

The present invention provides methods and assays for: (1) assessing the presence, viability and/or developmental stage of an embryo for use in an ART procedure, and (2) assessing the quality of, and improving, an ART protocol. The methods and assays are based on the identification of protein profiles and biomarkers for: (1) the secretome of an embryo, or (2) the proteins present in an embryo.

The protein profiles used in the methods and assays may be obtained via mass spectrometric analysis. The mass spectrometric data provide a spectrum of peaks, with each peak representing a peptide, protein or group of proteins present in the sample. For a given set of experimental conditions, a spectrum represents a protein profile for the group of proteins present in a sample.

Suitable mass spectrometric techniques for the study of proteins include, laser desorption ionization mass spectrometry and electrospray ionization mass spectrometry. Within the category of laser desorption ionization (LDI) mass spectrometry (MS), both matrix assisted LDI (MALDI) and surface assisted LDI (SELDI) time-of-flight (TOF) MS may be employed. SELDI TOF-MS is particularly well-suited for use in the present methods because it provides attomole sensitivity for analysis, quantification of low abundant proteins (pg-ng/ml) and highly reproducible results.

In a typical method in accordance with the present invention, a sample containing proteins is and subjected to mass spectrometric analysis. Depending upon the number and molecular weight range of the proteins present in a sample, it may be desirable to conduct a protein fractionation step on the sample in order to separate the protein content of the sample into separate fractions prior to mass spectrometric analysis. In such instances, each of the resulting fractions could be analyzed separately. In addition, the sample may require some pre-treatment in order to prepare it for a particular mass spectrometric protocol. For example, when SELDI TOF-MS is used, a sample containing proteins is placed on a protein chip and an energy absorbing matrix is applied to the sample on the chip so that laser energy applied to the sample ionizes proteins in the sample which enables the mass of the proteins to be measured using mass spectrometry. The protein chips generally include a solid substrate onto which an absorbent adapted to capture polypeptides is attached. Proteins chips for use with the present methods and assays are known and commercially available. Such protein chips include Ciphergen ProteinChip arrays available from Ciphergen Biosystems (Fremont, Calif.).

In a first aspect of the invention, protein profiles representing the secretome of viable or degenerating embryos are collected and biomarkers indicative or embryo viability or developmental stage are identified from the protein profiles. In this aspect of the invention, the samples from which the protein profiles and biomarkers are identified may be any samples that include the secretome of an embryo. Such samples may be any spent medium that includes proteins secreted by embryonic cells, including stem cells. For example, the samples may be media in which embryos have been prepared for in vitro fertilization, including embryo culture media, embryo transfer media and embryo cryopreservation media. Alternatively, the sample may be a biological medium or fluid, including placenta, and follicular, oviduct and uterine fluid samples that has come into contact with an embryo.

The embryos from which the protein profiles are identified may be in any of various stages of embryo development. As used herein, the term “embryo” is used broadly to include stages of development from a fertilized egg (zygote) to a fetus (e.g., a fetus in the first trimester of pregnancy). For example, the embryo may be a two-cell embryo, a four-cell embryo, an eight-cell embryo, a morula (16-32 cell embryo), or a blastocyst stage embryo. In some embodiments of the invention protein profiles are identified for an embryo, or embryos, over a range of different developmental stages. The embryos may be viable embryos or may be embryos undergoing degeneration.

The protein profiles may be stored, for example, in a database, including an electronic database. In one exemplary embodiment, a database of protein profiles may include protein profiles representative of embryos in a plurality of different developmental stages and/or protein profiles representative of different embryo morphologies. In one specific example, a protein profile database includes protein profiles representative of developing embryos and protein profiles representative of embryos in different stages of degeneration.

Once the protein profiles have been obtained, individual biomarkers and/or biomarker patterns indicative of embryo viability or developmental stage may be identified. As used herein, a biomarker indicative of embryo viability is a biomolecule (e.g., a polypeptide or protein) that is differentially expressed by the cells of a developing embryo as compared to the cells of a degenerating embryo. Similarly, a biomarker indicative of embryo developmental stage is a biomolecule (e.g., a polypeptide or protein) that is differentially expressed by the cells of an embryo in a first developmental stage as compared to the cells of an embryo in a second developmental stage. A biomolecule is differentially expressed if it is present at statistically significant elevated or decreases levels in the cells of a first sample as compared to the cells of a second sample.

In the present methods, a comparison of the protein profiles for embryos in different developmental stages, or for embryos having different morphologies, may be used to identify differentially expressed proteins, thereby identifying biomarkers. Importantly, by analyzing protein profiles, as opposed to individual proteins, biomarker patterns can be identified, where biomarker patterns are defined by a pattern of protein expression for a plurality of different proteins in a sample. The plurality of proteins that define the pattern make up a biomarker panel which provides a means for assessing embryo viability and/or developmental stage. Thus, in one embodiment of this aspect of the invention, protein profiles are obtained for the secretome of at least one viable embryo and for the secretome of at least one degenerating embryo. The protein profiles are then compared to identify individual biomarkers and biomarker patterns indicative of embryo viability. In some embodiments, protein profiles are obtained and biomarkers and biomarker patterns are identified for embryos in more than one stage of development. Suitable systems and methods for identifying differences in the expression of specific proteins based on mass spectrometric data are described in U.S. Patent Application Numbers 2004/0005634 and 2003/0134304, the entire disclosures of which are incorporated herein by reference.

Using the methods provided herein, as described in more detailed in Example 1 below, biomarker panels that include biomarkers indicative of embryo viability at different developmental stages have been identified. The inventors have discovered that these biomarkers are present in the secretome of a viable embryo, but absent in the secretome of a degenerate embryo. One such biomarker is ubiquitin. Other biomarkers include albumin fragments. Still other biomarkers are listed by their molecular weights, as measured by SELDI TOF-MS in accordance with the Example 1 below, in Table 1. Although the absolute identity of each biomarker is not known, such knowledge is unnecessary, because the biomarkers are sufficiently characterized by their mass and affinity characteristics to measure them in a patient. In should be noted, however, that the mass of each biomarker that makes up a biomarker panel may vary based on the particular state of the protein. For example, the mass of each individual biomarker will depend on whether the biomarker is modified, cleaved or phosphorylated. These variations in mass are reflected in the term “about” when used to describe the mass of each biomarker. In some instances, the term “about”, as it relates to biomarker mass, covers biomarkers whose mass varies by up to 200 Da, from the mass quoted in Table 1. This includes embodiments where the term “about”, as it related to biomarker mass, covers biomarkers whose mass varies by up to 100 Da from the mass quoted in Table 1, further includes biomarkers whose mass varies by up to 50 Da, from the mass quoted in Table 1, and still further includes biomarkers whose mass varies by up to 20 Da from the mass quoted in Table 1. Fortunately, any ambiguity that might be introduced by this variation in the mass of the biomarkers is mitigated by the use of biomarker panels to assess embryo viability and development. The use of a biomarker panel, as opposed to a single biomarker, is significant because it allows the user to monitor a qualitative pattern of biomarker expression, rather than requiring the user to definitively identify a single biomarker.

TABLE 1

| Developmental | |
|---|---|
| Stage | Protein/Biomarker MW (Da) (p < 0.05) |
| Day 2 | 2377, 8578, 13728, 9189, 4400, 6303, 3370, 3440, 3809, |
| | 3845, 3960, 4250, 4380, 4495, 4525, 4810, 4950, 5230, |
| | 5375, 5590, 5687, 5795, 6185, 6970, 7610, 8210, 8240, |
| | 8550, 8600, 9395, 9594 Da & 10.1, 10.7, 10.8, 10.9, |
| | 11.0, 11.1, 11.4, 14.6, 15.5, 15.8 & 16.7 kDa |
| Day 3 | 8573, 9180, 4400, 2771 (2658, 2788, 2977, 3103, 3118, |
| | 3302, 3578, 3985) 3370, 3440, 4525, 4810, 5220, 5230, |
| | 5370, 5785, 8208, 8163, 8190, 8222, 8550, 9168, 9180 |
| | Da & 10.7, 10.8, 11.3, 14.6, 15.8, 16.7, 18.6 kDa |
| Day 4 | 8573, 3118, 6183, 3836, 4302, 4520, 4800, 4954, 5370, |
| | 6223, 6295, 8190, 8200, 8785, 9080 Da & 10.7, 10.8, |
| | 11.3, 14.6, 15.8, 16.0, 16.7 kDa |
| Day 5 | 3941, 8576, 2888, 6231, 13680 (3580, 4494, 4041, |
| | 3837, 2792) 3995, 6235, 8190, 8775 Da & 10.7, 10.9, |
| | 11.3, 12.1, 15.8, 16.0, 16.7 kDa |

The biomarkers in Table 1 (except those in parentheses) are present in embryonic samples and absent in control samples. Without wishing or intending to be bound to any theory, the inventors believe the biomarkers shown in parentheses may represent peptides in the media that are utilized by the embryos and, hence, decrease in expression.

The protein profiles, biomarkers and biomarker patterns provided herein may be used in non-invasive clinical assays to assess the quality or developmental stage of an embryo produced in an ART procedure. In such an assay, a specimen containing spent medium from an ART protocol (e.g., an embryo culture medium) that contains, or is suspected of containing, proteins secreted by an embryo may be collected and a protein profile for the specimen obtained. The protein profile is then analyzed to look for biomarkers indicative of embryo viability. This analysis provides an assessment of embryo quality, where the identification of a greater number of biomarkers indicates a higher quality embryo. By assessing each of the embryos provided by in an ART procedure in this manner, the highest quality embryo or embryos may be identified.

A second aspect of the invention provides methods and assays for use in the development of optimized ART protocols. In this aspect of the invention, protein profiles representing the proteins present in embryos naturally produced in vivo are collected and biomarkers characteristic of these embryos are identified from the protein profiles. In this aspect of the invention, the samples from which the protein profiles and biomarkers are identified may be any samples that include the proteins present in an in vivo embryo. For example, the samples may be embryo tissue samples or biological fluid samples from an embryo.

Again, the embryos from which the protein profiles are identified may be in any of various stages of embryo development and the protein profiles may be stored, for example, in a database, including an electronic database.

Once the protein profiles have been obtained, individual biomarkers and/or biomarker patterns characteristic of embryos produced through natural reproduction may be identified. These biomarkers are than used in assays to assess the quality of an ART protocol. In such an assay, a sample containing the proteins present in an embryo produced through an ART procedure (e.g., embryonic tissue from a non-human mammal) may be collected and a protein profile for the specimen obtained. The protein profile is then analyzed to look for biomarkers characteristic of an embryo produced via natural reproduction. This analysis provides an assessment of the quality of the protocol used to produce the in vitro embryo, where the identification of a greater number of biomarkers indicates a protocol that better simulates the conditions of naturally occurring reproduction.

The protein profile for the in vitro embryo may be obtained from an embryo at any point during an ART protocol. By way of non-limiting example, the protein profile could be obtained for an in vitro embryo that has undergone cryopreservation (e.g., vitrification or freezing), undergone transfer, or undergone embryo culture. Similarly, the protein profile for the in vivo embryo may be obtained from an embryo at various points during a pregnancy.

When one or more biomarkers characteristic of an embryo naturally produced in vivo are found to be lacking in the protein profile for an in vitro embryo, the processing parameters (e.g., media or conditions) are altered to reduce, minimize or eliminate the differences between the protein profiles for the in vivo and in vitro embryos. This process may be a multi-step process where one parameter of an ART protocol (e.g., the nature or amount of one component of a embryo processing medium (e.g., an embryo culture medium, an embryo transfer medium, or an embryo cryopreservation medium), or one aspect of the processing conditions (e.g., temperature, pressure, pH), is altered at a time until a comparison of the protein profiles for the in vivo embryo and the in vitro embryo indicate that the medium and/or conditions have been optimized.

In one variation of the second aspect of the invention, protein profiles representing the proteins present in naturally produced gametes, such as oocytes, are collected and biomarkers characteristic of the gametes are identified from the protein profiles. These protein profiles and biomarkers may be used to optimize ART procedures, using comparisons with ART-produced gametes (e.g., oocytes produced by stimulation protocols), using methods and assays analogous to those described above with respect to the in vivo and in vitro embryos. Such assays allow for the optimization of stimulation protocols for the maturation of oocytes in the ovaries.

The embryos used in the present methods may be embryos from animals such as mammals including the human. Suitable mammals include, but are not limited to, primates such as, but not limited to lemurs, apes, and monkeys; rodents such as rats, mice, and guinea pigs; rabbits and hares; cows; horses; pigs; goats; sheep; marsupials; and carnivores such as felines, canines, and ursines. In some embodiments, the embryos are human embryos. In other embodiments, the embryos are mouse embryos. In some embodiments, the embryos are non-human embryos.

The invention will be better understood from the Examples which follow. However, one skilled in the art will readily appreciate that the specific compositions, methods and results discussed are merely illustrative of the invention and no limitations on the invention are implied.

### Examples

### Example 1

### Identification of Biomarkers Indicative of Embryo Viability

Twenty eight human embryos and 64 mouse embryos were cultured in sequential media (G1/G2) supplemented with human serum albumin or recombinant human albumin at 37° C., 5% O₂ & 6% CO₂. Embryos were moved to fresh 10 μl drops of media every 24 h and 5 μl of spent media were subsequently processed and analyzed by mass spectrometry to determine the embryo secretome at each developmental stage up to the hatching blastocyst. Culture media was applied directly to the protein chip surfaces without any sample pre-treatment. The chips were washed in the appropriate binding buffer prior to incubation of the samples (5 μl) in a humidified chamber for 30 minutes. After incubation the samples are removed from the chips and a series of binding buffer and water washes followed. The chips were then left to dry completely before the addition of 2×1 μl of an energy absorbing solution (SPA in 50% acetonitrile, 0.5% trifluoroacetic acid). Chips were measured in a SELDI-TOF mass spectrometer (Enterprise 4000 Series, Ciphergen) according to an optimized protocol using protein chip software 3.1 (Ciphergen).

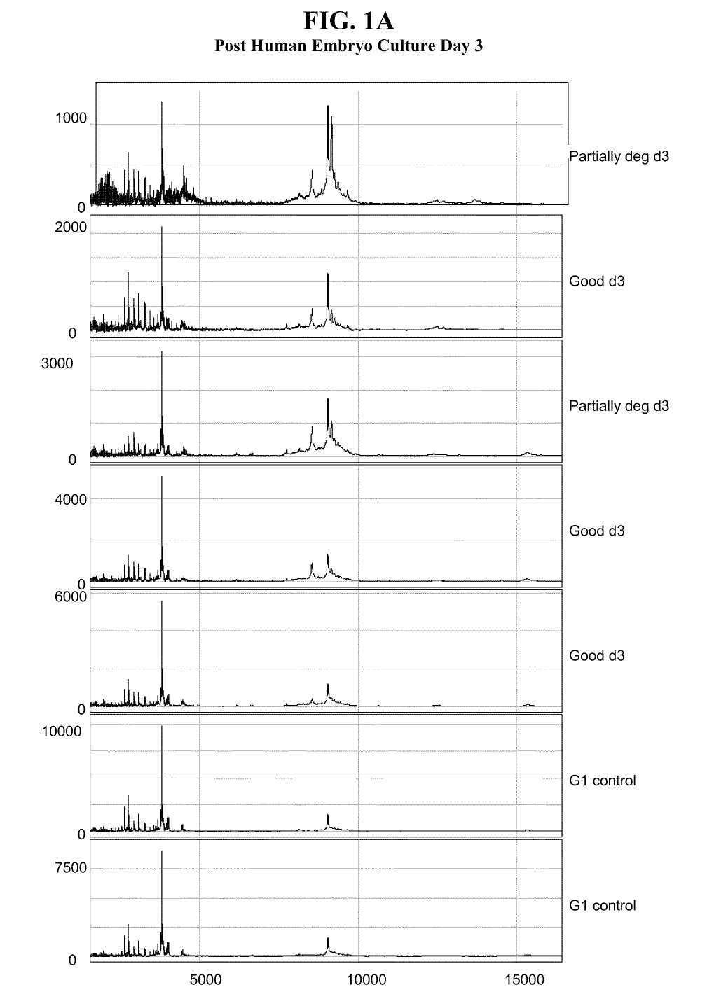

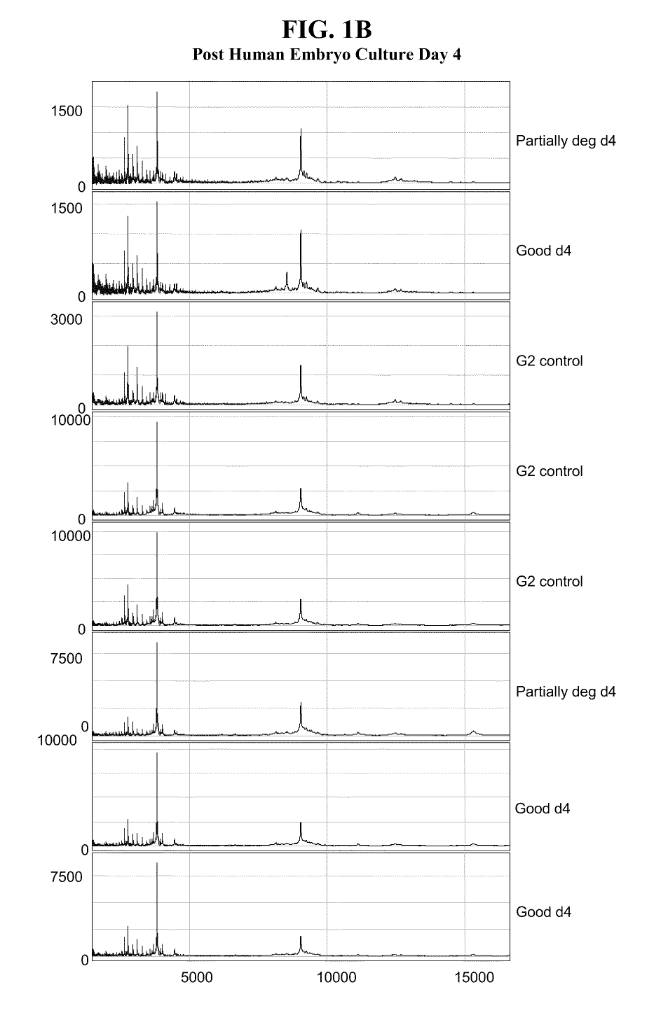

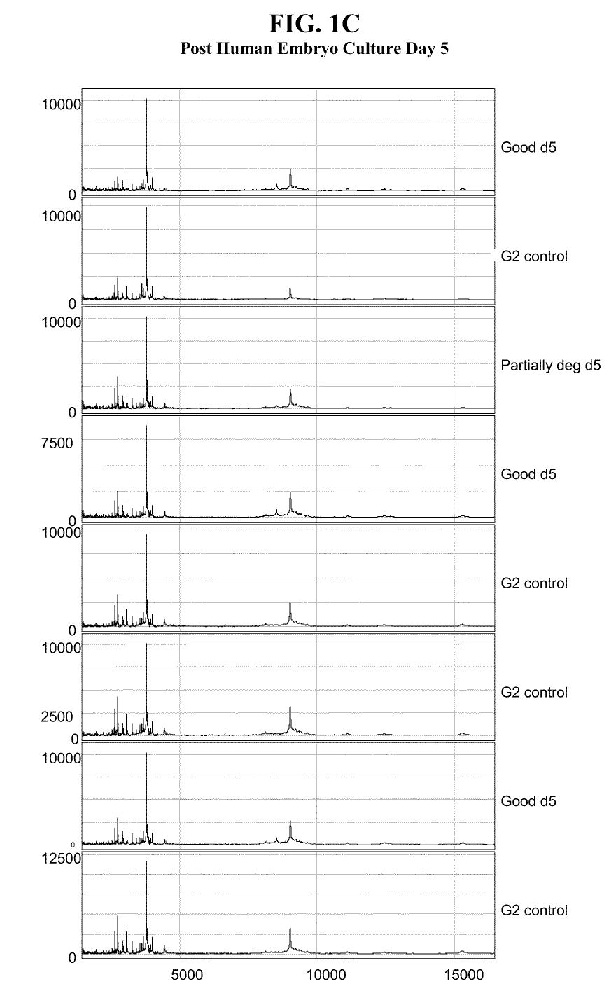

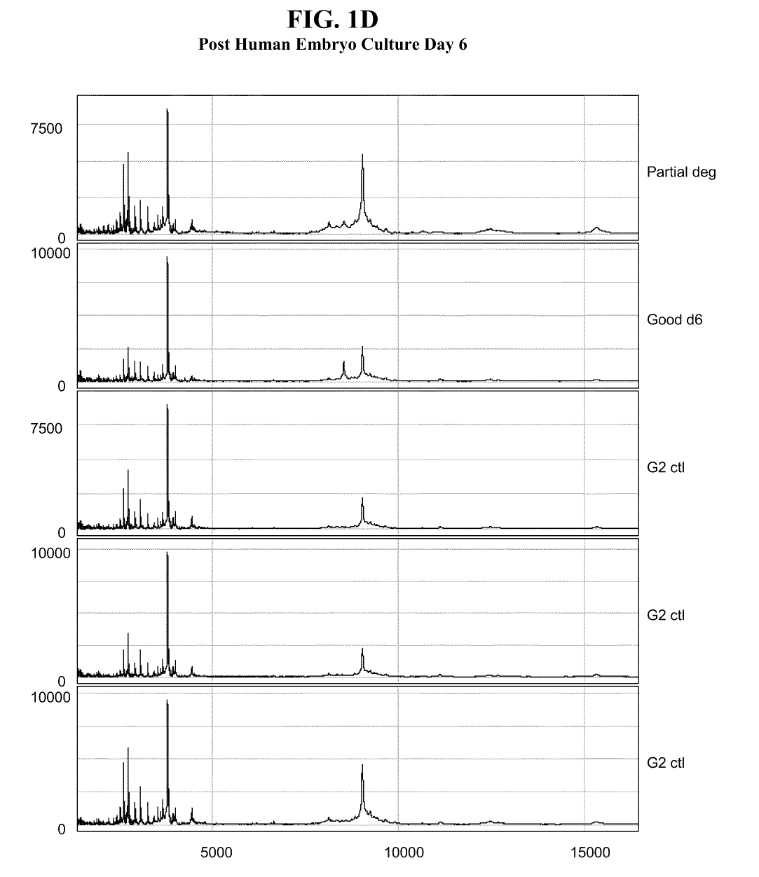

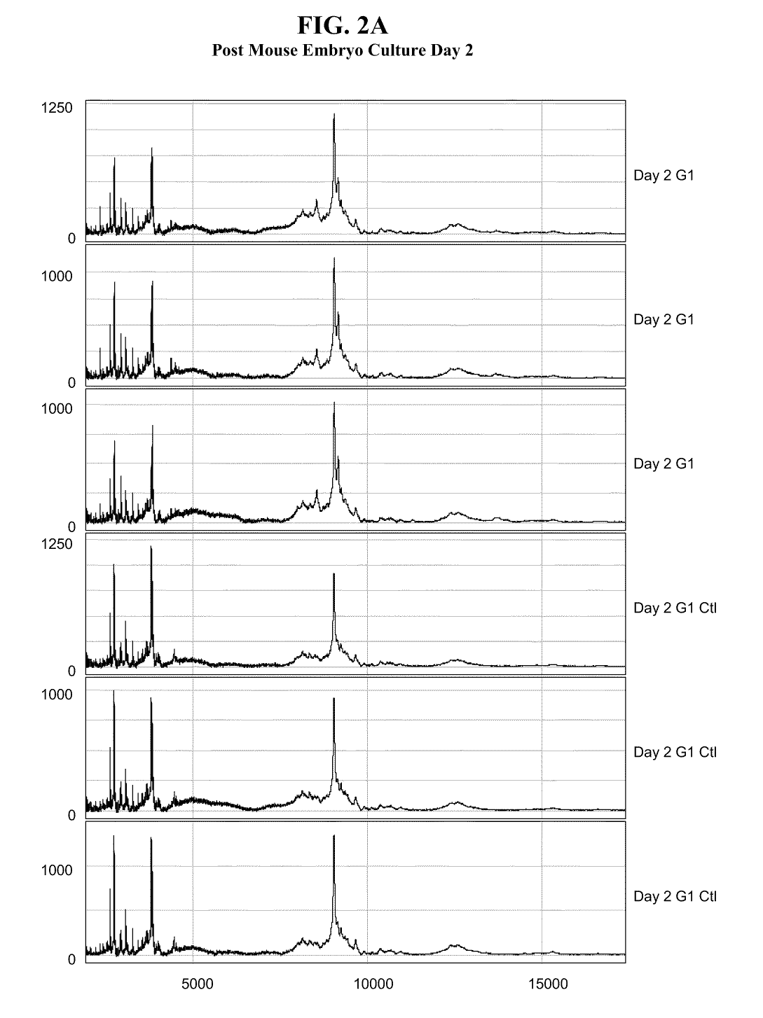

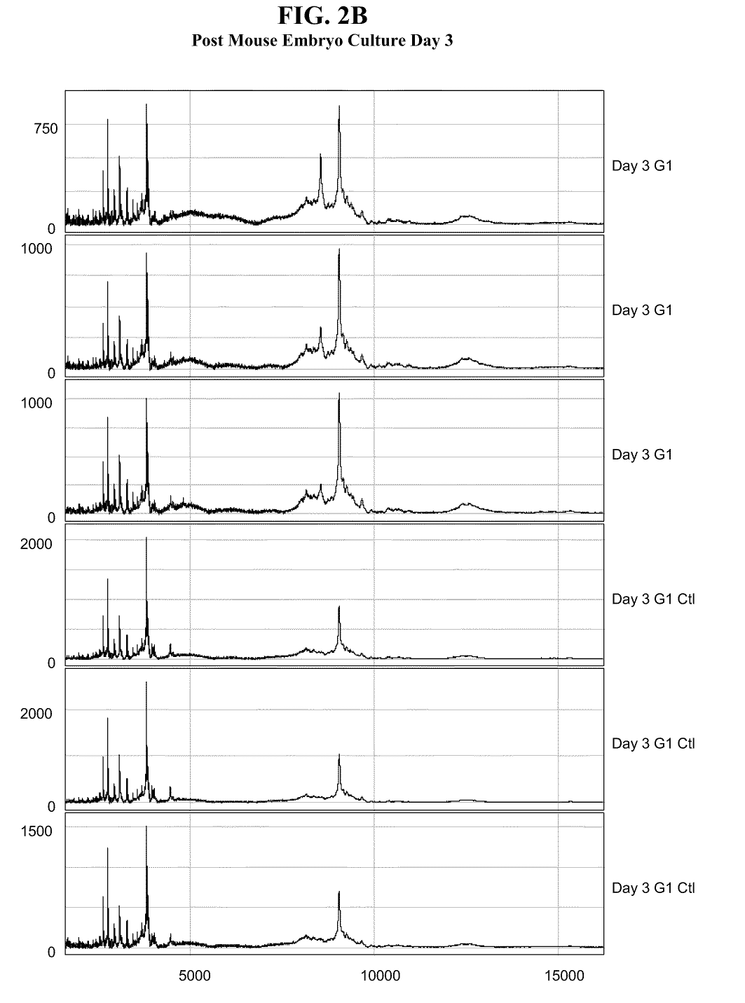

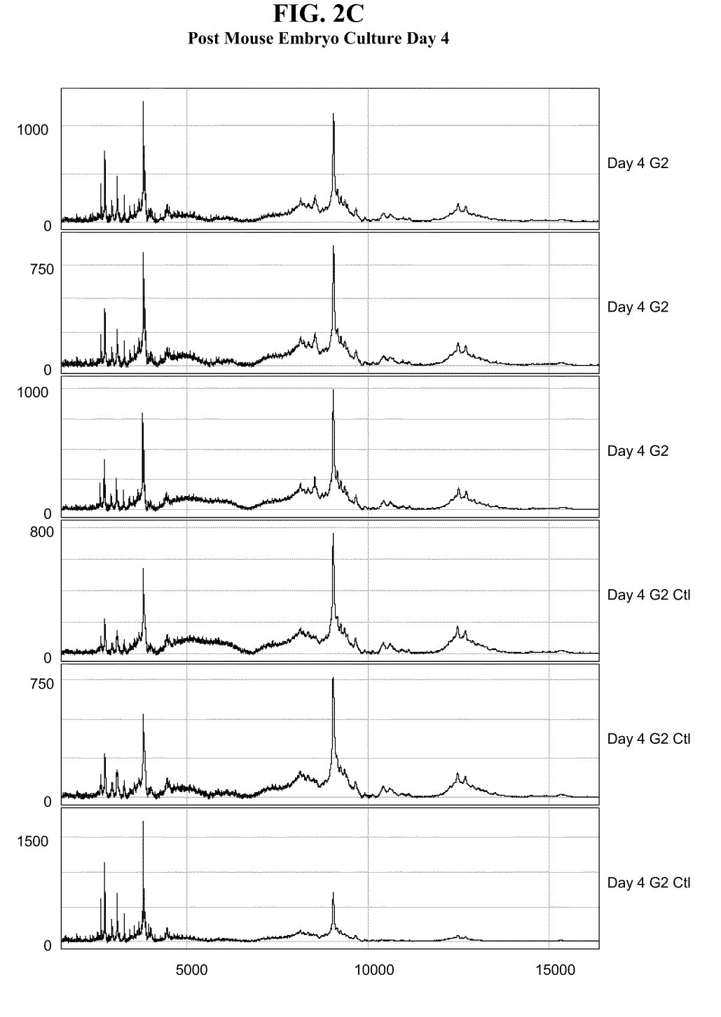

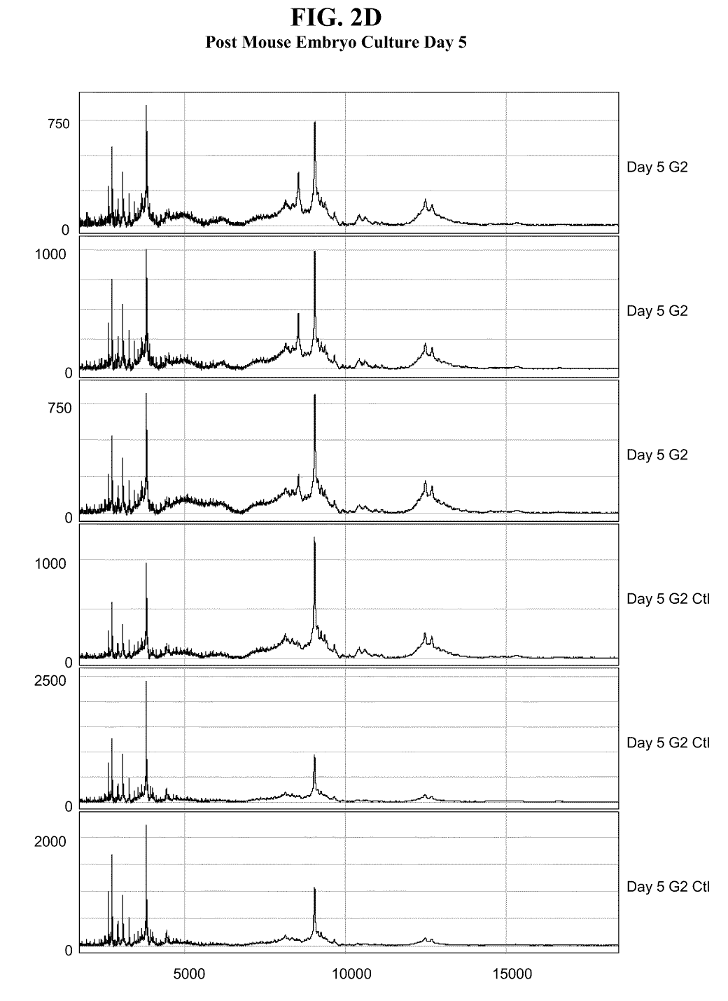

Media samples cultured with and without the presence of embryos revealed specific proteins and biomarkers that are produced by these embryos in culture (p<0.05). These experiments have confirmed a protein profile that consistently detects the presence of cultured embryos. Unique secretome profiles were identified between embryos of different morphologies. Significantly, several proteins (2 to 14 kDa) unique to the secretome of developing embryos were observed for both the human (FIG. 1A-D) and mouse (FIG. 2A-D). At the first cleavage stage of mouse embryonic development an albumin fragment (2.4 kDa) is significantly observed in the protein profile. The protein produced in highest abundance by the human preimplantation embryo was ubiquitin (8.5 kDa). Production of ubiquitin by the cleavage stage embryo did not appear to be related to morphology. However, from the early to the hatching blastocyst stage ubiquitin production was associated with morphology. Human and mouse embryos undergoing degeneration exhibited a unique secretome at all developmental time points. The significant proteins/biomarkers that were observed for developing embryos, but not for degenerate embryos, at each developmental stage are listed in Table 1. This panel of differently expressed proteins including quantification of ubiquitin production by the human embryo could form the base of a non-invasive assay to determine embryo viability. It should be noted that, although the results above allow viable and degenerate embryos to be distinguished based on the presence or absence of specific proteins, the quality of an embryo may also be assessed by looking at the level of expression of one or more biomarkers indicative of viability, where a reduction in embryo quality corresponds to a reduction in the expression of one or more of the biomarkers. By correlating the level of expression of one or more of the biomarkers with a desired success rate (e.g., as measured by successful IVF births), a threshold level of expression that marks an embryo as viable can be identified.

### Example 2

### Identification of Biomarkers Indicative of ART Quality

Embryos were cultured in sequential media (G1/G2) supplemented with human serum albumin or recombinant human albumin at 37° C., 5% O₂ & 6% CO₂. After culture the resultant embryos were extracted in groups of up to 5, processed and analyzed by mass spectrometry. To diminish protein-protein interactions, cell lysates were denatured in 9M urea, 2% 3-[(3-cholamidopropyl)dimethylammonio]-1-propanesulfonate (CHAPS) and 50 mM TrisHCl (pH 9) buffer and then diluted into the appropriate binding buffers for each chromatographic array before direct application to the protein chip surfaces. Samples were incubated on the chips in a humidified chamber for 30 minutes before removal and a series of binding buffer and water washes. The chips are left to dry completely before the addition of 2×1 μl of an energy absorbing solution (SPA in 50% acetonitrile, 0.5% trifluoroacetic acid). Protein chips were measured in a SELDI-TOF mass spectrometer (Enterprise 4000 Series, Ciphergen) according to an optimized protocol using protein chip software 3.1 (Ciphergen).

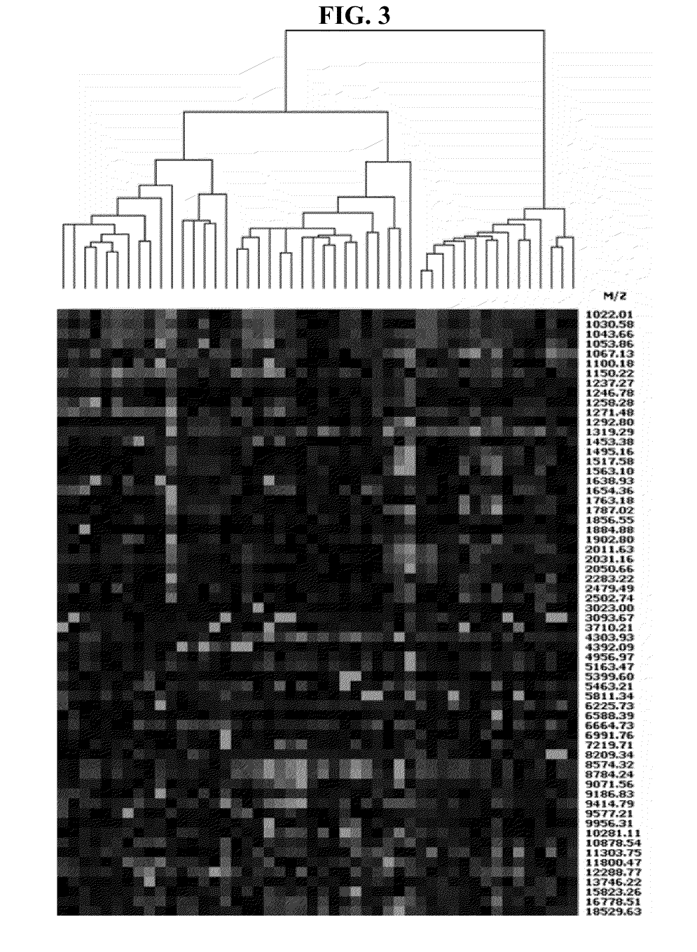

Novel biomarkers were observed at specific developmental time points with both down and up regulation of protein production from zygote to blastocyst (n>30) (p<0.05). In addition, a panel of statistically significant novel biomarkers distinguished in vivo embryos from in vitro embryos cultured in G1/G2 sequential media at 5% O₂ and 6% CO₂. Each of these biomarkers is represented in the heat map of FIG. 3. These novel biomarkers may allow for the improvement of better in vitro culturing conditions, stimulation protocols, and cryopreservation, thereby increasing the success rates of human IVF. For example, embryos cultured under high oxygen (20%) showed consistent down-regulation of 10 proteins between 4,000 to 20,000 Dalton (p<0.05), while the protein profiles of embryos cultured under low oxygen (5%) conditions were more comparable to the in vivo state.

The present invention is not to be limited in terms of the particular embodiments described in this application, which are intended as single illustrations of individual aspects of the invention. Many modifications and variations of this invention can be made without departing from its spirit and scope, as will be apparent to those skilled in the art. Functionally equivalent methods and apparatuses within the scope of the invention, in addition to those enumerated herein, will be apparent to those skilled in the art from the foregoing descriptions. Such modifications and variations are intended to fall within the scope of the appended claims. The present invention is to be limited only by the terms of the appended claims, along with the full scope of equivalents to which such claims are entitled. It is to be understood that this invention is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

Other embodiments are set forth within the following claims.

What is claimed is:

1. A method for assessing the viability or developmental stage of an embryo in a sample, the method comprising:
(a) analyzing a sample having or suspected of having proteins secreted from embryonic cells to identify a protein profile for the secreted proteins; and
(b) identifying at least two biomarkers selected from the biomarkers listed in the following Table:

| Developmental | |
|---|---|
| Stage | Biomarker MW |
| Day 2 | 2377, 8578, 13728, 9189, 4400, 6303, 3370, 3440, 3809, |
| | 3845, 3960, 4250, 4380, 4495, 4525, 4810, 4950, 5230, |
| | 5375, 5590, 5687, 5795, 6185, 6970, 7610, 8210, 8240, |
| | 8550, 8600, 9395, 9594 Da & 10.1, 10.7, 10.8, 10.9, |
| | 11.0, 11.1, 11.4, 14.6, 15.5, 15.8 & 16.7 kDa |
| Day 3 | 8573, 9180, 4400, 2771 (2658, 2788, 2977, 3103, 3118, |
| | 3302, 3578, 3985) 3370, 3440, 4525, 4810, 5220, 5230, |
| | 5370, 5785, 8208, 8163, 8190, 8222, 8550, 9168, 9180 |
| | Da & 10.7, 10.8, 11.3, 14.6, 15.8, 16.7, 18.6 kDa |
| Day 4 | 8573, 3118, 6183, 3836, 4302, 4520, 4800, 4954, 5370, |
| | 6223, 6295, 8190, 8200, 8785, 9080 Da & 10.7, 10.8, |
| | 11.3, 14.6, 15.8, 16.0, 16.7 kDa |
| Day 5 | 3941, 8576, 2888, 6231, 13680 (3580, 4494, 4041, 3837, |
| | 2792) 3995, 6235, 8190, 8775 Da & 10.7, 10.9, 11.3, |
| | 12.1, 15.8, 16.0, 16.7 kDa |

wherein the biomarkers are indicative of embryo viability for an embryo at the developmental stage listed in the table.

2. The method of claim 1, further comprising comparing the quantity of the at least two biomarkers in the sample to the quantity of the biomarker in a control sample known to lack favorable developmental potential, wherein a higher level of expression of the at least two biomarkers in the test sample compared to the control sample indicates a diagnosis of favorable developmental potential.

3. The method of claim 1, further comprising comparing the quantity of the at least two biomarkers in the sample to the quantity of the biomarker in a control sample known to lack favorable developmental potential, wherein a higher level of expression of the at least two biomarkers in the test sample compared to the control sample indicates a diagnosis of favorable IVF pregnancy rate.

4. The method of claim 1, wherein the embryo is produced in vivo through natural reproduction.

5. The method of claim 1, wherein the embryo is produced in vitro through assisted reproduction technology.

6. The method of claim 1, wherein the sample is a biological medium or fluid that has come into contact with an embryo.

7. The method of claim 1, wherein the sample is spent media from a pre-implantation IVF procedure.

8. The method of claim 1, wherein at least one additional biomarker is selected from the group ubiquitin, albumin, and fragments thereof.

9. The method of claim 8, wherein the ubiquitin or fragment thereof is an 8.5 kDa fragment.

10. The method of claim 8, wherein the albumin or fragment thereof is a 2.4 kDa fragment.

11. The method of claim 1, wherein at least one additional biomarker is a protein having a molecular weight of about 16.7 kDa.

12. The method of claim 1, wherein the analyzing is by mass spectrometry.

13. The method of claim 12, wherein the analyzing is by SELDI-TOF.

14. The method of claim 1, wherein at least three biomarkers indicative of embryo viability are identified.

15. The method of claim 1, wherein at least four biomarkers indicative of embryo viability are identified.
